(19) United States
(12) Patent Application Publication
(10) Pub. No.: US 2020/0069675 A1
(43) Pub. Date: Mar. 5, 2020

(54) PHARMACEUTICAL PREPARATIONS OF SEBACOYL DINALBUPHINE AND ACETAMINOPHEN AND METHODS FOR TREATING PAIN

(71) Applicant: International Education Foundation, New Taipei City (TW)

(72) Inventors: Oliver Yoa-Pu HU, Taipei City (TW); Yen-Lun CHEN, Tainan City (TW)

(73) Assignee: International Education Foundation, New Taipei City (TW)

(21) Appl. No.: 16/120,346

(22) Filed: Sep. 3, 2018

(51) Int. Cl. A61K 31/485; A61K 31/167; A61P 25/04

(52) U.S. Cl. CPC A61K 31/485; A61P 25/04; A61K 31/167

(57) ABSTRACT

The present invention relates to pharmaceutical compositions/combination/kit and methods for treating pain, which provide synergistic analgesic effects and less side effects.

### TECHNOLOGY FIELD

This invention is related to a novel pharmaceutical combination of compounds having synergistic analgesic activity. The present invention also relates to pharmaceutical preparations and methods for treating pain, particularly providing enhanced analgesic effects.

### BACKGROUND OF THE INVENTION

A number of drugs have been developed for treating pain. However, side effects need to be solved. Recent reports which published in the journal Proceedings of the National Academy of Sciences of the United States of America (PNAS), men who take the non-aspirin nonsteroidal anti-inflammatory drugs (NSAIDs), ibuprofen, not only could increase heart attack and stroke (FDA warning) for months at a time, but could also be putting their fertility at risk. Acetaminophen is not a non-aspirin nonsteroidal anti-inflammatory drugs (NSAIDs) but has side effects of hepatotoxicity or nephrotoxicity.

In addition, overuse of highly addictive opioids has led to a health crisis across the world, especially in the US where more than 60,000 people died after overdoses in 2016 alone. “Tens of thousands of people are dying every year in the US because of opioid overdoses; in the last year more than 50,000 people died. That is as many as died in the Vietnam War in the US.

New type of opiods drugs such as nalbuphine, buprenorphine, butorphanol, so-called narcotic agonist-antagonist analgesics have been developed. They exhibit a dual action of agonist and antagonist on opiods-receptors as reported by Schmidt, W. K. et al (Drug Alcohol Depend. 14, 339, 1985; British Journal of Pain. 6, 11-16, 2012), where pointed out that dual action of those drugs not only had high affinity to opium receptor but also served as anatagonist. For example, nalbuphine was the antagonist for Mu receptor and agonist for Kappa receptor. Those agonistiantagonist drugs have improvement on untoward effects of opiods drugs, such as addiction and respiratory suppression. Nalbuphine is the most widely used one and has excellent therapeutic efficacy. After continuous use ofnalbuphine for 6 months, no significant addiction and addition was found. Those narcotic agonist-antagonist analgesics exhibits only slight respiratory inhibition. In clinical use, nalbuphine is safer than the traditional narcotic analgesics and classified as narcotics slush (Drug Alcohol Depend. 14, 339, 1985; Anaesthesist. 63, 135-143, 2014).

Nalbuphine is a synthetic agonist-antagonist that is chemically related to both naxloxone, a narcotic antagonist, and oxymorphone, a potent narcotic analgesic. Action of nalbuphine at the kappa-receptors produce alternations in the perception of pain as well as the emotional response to pain, possibly by altering the release of neurotransmitters from afferent nerves sensitive to painful stimuli. Oral nalbuphine has been shown to be only one quarter to one fifth as potent as intramuscular nalbuphine as a postoperative analgesic. The conventional form of nalbuphine is not practical for oral administration, because the bioavailability through oral administration is less than 5%, as described in Br J Clin Pharmacol 1988; 25:264-8.

This double-blind, randomized, parallel, placebo-controlled study evaluated the analgesic effects of single oral doses of nalbuphine, acetaminophen, and the contribution of each to the efficacy of their combination in 128 hospitalized patients with postoperative pain. Subjective reports of patients evaluated each hour for 6 hours were used as indices of analgesic response. Both nalbuphine alone and acetaminophen alone were significantly superior to placebo for most measures of total and peak analgesia. However, the combination of nalbuphine and acetaminophen was not significant for any analgesic measurements, indicated the combination just have the additive effect of the components, as described in CLIN PHARMACOL THER 1986; 39:295-9.

Sebacoyl dinalbuphine (SDE) oil solution is a pharmaceutically acceptable long acting dosage forms, is administered once a day, or once for several days. Even when large amounts are administered, the occurrence of untoward effects were minimized. The advantage of SDE are long duration, untoward effects, and safety that should improve therapeutic quality. The dosing interval can be set up to 7 days instead of 3-5 hours for post-operation patient. For last cancer stage patient's administration of the present invention dosage forms, instead of hospitalization can be given the same therapeutic efficacy.

An ideal analgesic should exhibit short onset time, long acting, potent, no addiction, no or minimum respiratory inhibition and should have few adverse effects. Due to the current worldwide opioids crisis, there is an obvious need to provide novel pharmaceutical preparations and methods for treating pain, especially moderate to severe pain without addiction, respiratory depression, with short onset time, long duration and less side effects, through new discovery including combination with better results such as synergic effects.

### BRIEF DESCRIPTION OF THE DRAWINGS

The embodiment is shown below to illustrate the present invention. It should be understood, however, that the present invention is not limited to the preferred embodiment shown. In the drawing:

FIG. 1 shows the analgesic profile in SD rats following oral administration of SDE 75 mg/kg, AAP 100 mg/kg and combo (SDE+AAP) with by a standard paw pressure test.

FIG. 2 Analgesic profile in SD rats following oral administration either NAL 60 mg/kg, AAP 100 mg/kg and combinations oral administration by paw pressure in SD rats.

### BRIEF SUMMARY OF THE INVENTION

The present invention provides a novel pharmaceutical preparation and method for treating pain. Specifically, the pharmaceutical preparation comprises sebacoyl dinalbuphine or its metabolites or derivatives and/or acetaminophen (AAP) or its derivatives, together with one or more pharmaceutically acceptable excipients. The pharmaceutical preparation of the present invention can provide summation/synergic effects of pain relief, improved onset time (shorter), duration of action, oral bioavailability (AUC) and peak of maximum.

In some embodiments, the pharmaceutical preparation of the present invention comprises a therapeutically effective amount of a first analgesic agent which is sebacoyl dinalbuphine or its metabolites or derivatives and/or a therapeutically effective amount of a second analgesic agent which is acetaminophen (AAP) or its derivatives, together with one or more pharmaceutically acceptable excipients.

In some embodiments, the pharmaceutical preparation of the present invention comprises
(i) a first analgesic composition comprising a therapeutically effective amount of a first analgesic agent which is sebacoyl dinalbuphine or its metabolites or derivatives; and
(ii) a second analgesic composition comprising a therapeutically effective amount of a second analgesic agent which is acetaminophen or its derivatives.

In some embodiments, the first analgesic agent is sebacoyl dinalbuphine in the free base form or a pharmaceutically acceptable salt.

In some embodiments, the first analgesic agent is sebacoyl dinalbuphine ester (SDE), for example, as described in U.S. Pat. No. 6,225,321.

In some embodiments, the first analgesic agent is in an amount effective to act as a bioavailability enhancer of the first analgesic agent.

In some embodiments, the second analgesic agent is acetaminophen (AAP), for example, as described in U.S. patent application Ser. No. 14/441,317 (US20170172950A1).

In some embodiments, the excipient as used herein is selected from the group consisting of Eudragit S100), dicalcium phosphate dehydrate, Pluronic F68, hexitol, Crospovidone, Sodium starch glycolate, Aerosil 200, trichlorosucrose, menthol, Saccharin, Sodium benzoate, Glyceryl behenate, Sodium lauryl sulfate. Providone K30, and any combination thereof.

In some embodiments, the first analgesic composition is formulated as an extended form and the second analgesic composition is formulated as an immediate release form.

In another aspect, the present invention provides a method for treating pain in a subject in need comprising administering to the subject an analgesic pharmaceutical preparation or specifically an analgesic pharmaceutical combination as described herein. Also provided is use of such analgesic pharmaceutical preparation or analgesic pharmaceutical combination as described herein for manufacturing a medicament for treating pain in a subject in need.

The details of one or more embodiments of the invention are set forth in the description below. Other features or advantages of the present invention will be apparent from the following detailed description of several embodiments, and also from the appending claims.

### DETAILED DESCRIPTION OF THE INVENTION

Unless defined otherwise, all technical and scientific terms used herein have the same meanings as is commonly understood by one of skill in the art to which this invention belongs.

As used herein, the articles “a” and “an” refer to one or more than one (i.e., at least one) of the grammatical object of the article. By way of example, “an element” means one element or more than one element.

The term “comprise” or “comprising” is generally used in the sense of include/including which means permitting the presence of one or more features, ingredients or components. The term “comprise” or “comprising” encompasses the term “consists” or “consisting of.”

The present invention provides a novel pharmaceutical preparation and method for treating pain. Specifically, the pharmaceutical preparation of the present invention comprises nalbuphine or its derivatives and/or acetaminophen or its derivatives, together with one or more pharmaceutically acceptable excipient. The pharmaceutical preparation of the present invention can provide summation/synergic effects of pain relief, improved oral bioavailability and less side effects.

As used herein, the term “pharmaceutical preparation” can refer to pharmaceuticals in any forms, for example, a composition, a combination or a kit. A composition can refer to a homogenous mixture, for example, in a form e.g. tablets, capsules, pills, powders, granules, solutions, suspensions and emulsions and any pharmaceutical acceptable forms. A combination can refer to a product obtained from combining two or more active ingredients which are present physically separately in one or more packaging units for time-sequential administration. A kit can refer to a collection or set of the aforementioned pharmaceutical preparation, preferably, provided in separate form within a single container. The container, also preferably, comprises instructions for using such pharmaceutical preparation or carrying out the methods of the present invention.

As used herein, the term “nalbuphine (NAL)” is intended to include nalbuphine itself and the chemical derivatives of the nalbuphine structure having equivalent pharmaceutical effect, including nalbuphine in the free base form or a pharmaceutically acceptable salt (except for nalbuphine hydrochloride) or ester of nalbuphine (including a monoester or a polyester such as sebacoly dinalbuphine ester (SDE), for example, as described in U.S. Pat. No. 6,225,321, the entire content of which is incorporated herein by reference).

As used herein, the term “acetaminophen (AAP)” is intended to include acetaminophen itself and the chemical derivatives of the acetaminophen structure having equivalent pharmaceutical effect, for example, as described in U.S. patent application Ser. No. 14/441,317 (US20170172950A1), the entire content of which is incorporated herein by reference.

According to the present invention, an analgesic pharmaceutical preparation as described herein may comprise a therapeutically effective amount of a first analgesic agent which is nalbuphine or its derivatives and/or a therapeutically effective amount of a second analgesic agent which is acetaminophen (AAP) or its derivatives.

Preferably, the first analgesic agent or the second analgesic agent, as described herein, is present in a form of a composition formulated with one or more pharmaceutically acceptable excipients.

In some embodiments, the excipient as used herein is selected from the group consisting of Eudragit S100, dicalcium phosphate dehydrate, Pluronic F68, hexitol, Crospovidone, Sodium starch glycolate, Aerosil 200, trichlorosucrose, menthol, Saccharin, Sodium benzoate. Glyceryl behenate, Sodium lauryl sulfate, Providone K30, and any combination thereof.

In some embodiments, the pharmaceutical preparation of the present invention comprises a combination of a first analgesic agent (NAL or its derivatives) and a second analgesic agent (AAP or its derivatives) as described herein. In some embodiments, the first analgesic agent is in an amount of 1 mg or more, 5 mg or more, 10 mg or more, 20 mg or more, 30 mg or more, 50 mg or more. 75 mg or more, per dose. In certain embodiments, the second analgesic agent is in an amount of 100 mg or more, 200 mg or more, 300 mg or more, 500 mg or more, 750 mg or more, 900 mg or more, 1000 mg or more, per dose. In some embodiments, the second analgesic agent and the first analgesic agent (AAP or its derivatives: NAL or its derivatives) are present in a weight ratio of 1-1,000:1 or more (e.g. about 1.5:1, 5:1, 10:1, 25:1, 50:1, 75:1; 100:1.200:1, 300:1, 500:1, or 1.000:1).

According to the present invention, the pharmaceutical preparation provides a synergic analgesic effect of pain relief.

As used herein, a synergistic effect for example refers to simultaneous actions of separate factors or active agents which have a greater total effect than the sum of the individual factor effects.

In some embodiments, the pharmaceutical preparation comprising a first analgesic agent and a second analgesic agent as described herein provides a faster onset of analgesic effect and/or a longer duration of analgesic effect, when compared to the first analgesic agent or the second analgesic agent alone. In some embodiments, the faster onset of analgesic effect can be meant to achieve analgesic effects within 30 min after administration e.g. less than 25 min, 20 min or 15 min. In some embodiments, the longer duration of analgesic effect can be meant to sustain analgesic effects for 30 min or longer, e.g. 40 min or longer, 50 min or longer, 60 min or longer, 70 min or longer, 80 min or longer, 90 min or longer, or 100 min or longer.

In some embodiments, the pharmaceutical preparation comprising a first analgesic agent and a second analgesic as described herein provides an increased level of one or more pharmacokinetic parameters (e.g. AUC, Tₘₐₓ) of the first analgesic agent and the second analgesic agent in said pharmaceutical preparation, when compared to a respective value of the first analgesic agent in the first analgesic composition or the second analgesic agent in the second analgesic composition. For example, the value of a certain pharmacokinetic parameter of the pharmaceutical preparation comprising a first analgesic agent and a second analgesic as described herein may be at least 20% higher (e.g., 30% higher, 50% higher, 1-fold higher, 2-fold higher, or above) than that of the first analgesic agent in the first analgesic composition or the second analgesic agent in the second analgesic composition.

In some embodiments, the side effect includes nephrotoxicity and/or hepatotoxicity caused by the first analgesic agent and/or the second analgesic agent. In some embodiments, the side effect includes respiratory depression or addiction risk.

An increased level of an index or condition of toxicity may be used as an indicator of induction or occurrence of the toxicity (a toxic state) which is compared with reference to a control (or normal) level thereof. As used herein, a “normal level” or “control level” is meant to describe a value within an acceptable range of values that one of ordinary skill in the art and/or a medical professional e.g. a doctor would expect a healthy individual or population of similar physical characteristics and medical history to have. A “decreased” level of an index or condition of toxicity can be used as an indicator of reduction or removal of toxicity when compared with that of a corresponding toxic state. Especially, when a decreased level of an index or condition of toxicity comes close to or even becomes lower than a normal level or control level, the toxicity can be considered “eradicated.”

As used herein, the toxicity such as nephrotoxicity and/or hepatotoxicity can be caused by overdose of AAP. Overdose can refer to administration of a dose greater than a useful or standard dose that is an effective dose approved by a drug regulatory authority such as Food & Drug Administration or prescribed by a physician for treatment or prevention of a diseases condition or relief of symptoms thereof. For example, paracetamol tablets are the currently AAP drugs approved in the market for oral administration, the standard dose of which is 500 mg to 1 g paracetamol taken every 4-6 hours as required, up to a maximum of 4 g daily, for a human adult. Overdose of AAP can mean a dose greater than a useful or standard dose of AAP, for example, by 5%, 10%, 20%, 30%, 50%, 75%, 100% or more.

As used herein, the term “treating” refers to the therapeutic measures to a disease or the symptoms or conditions of a disease, which include but are not limited to applying or administering one or more active agents to a subject suffering from the disease or the symptoms or conditions of the disease or exacerbation of the disease. The purpose of the therapeutic measures is to treat, cure, mitigate, relieve, alter, remedy, ameliorate, improve, or affect the disease, the symptoms or conditions of the disease, disability caused by the disease, or exacerbation of the disease. Specifically, the present invention provides a pharmaceutical combination and method for treating pain.

As used herein, the term “individual” or “subject” includes human or non-human animals, in particular mammal, for example, companion animals (such as dogs, cats and the like), farm animals (such as cattle, sheep, pigs, horses, etc.), or laboratory animals (such as rats, mice, guinea pigs, etc.).

As used herein, the term “effective amount” refers to the amount of an active ingredient achieving desired biological efficacy or therapeutic effects in a subject being treated, for example, pain relief.

For the purpose of transport and uptake, an effective amount of an active ingredient according to the present invention may be formulated with a pharmaceutically acceptable excipient to form a suitable form of a pharmaceutical preparation. According to the routes of administration, the pharmaceutical composition of the present invention preferably comprise from about 0.1% to about 100% by weight of the active ingredient, based on the total weight of the composition. As used herein, the term “pharmaceutically acceptable” means that the carrier is compatible with the active ingredient of the composition (and does not affect the effect of the active ingredient), and, preferably, the carrier may stabilize the active ingredient and is safe for the subjects being treated. Such carrier may be a diluent, vehicle, excipient, or matrix to the active ingredient. Some examples of appropriate excipients include lactose, dextrose, starch, Arabic gum, gelatin, calcium silicate, microctystalline cellulose, sterilized water, syrup, and methylcellulose. The composition may additionally comprise lubricants, such as talc, magnesium stearate, and mineral oil; wetting agents; emulsifying and suspending agents; preservatives, such as methyl and propyl hydroxybenzoates, sweeteners; and flavoring agents. The composition of the present invention can provide the effect of rapid, continued, or delayed release of the active ingredient after administration to the patient. According to the present invention, the form of said composition may be tablets, pills, powder, lozenges, packets, troches, elixers, suspensions, lotions, solutions, syrups, soft and hard gelatin capsules, suppositories, sterilized injection fluid, and packaged powder.

The pharmaceutical preparation of the present invention may be delivered via any physiologically acceptable route, such as oral, parenteral (such as intramuscular, intravenous, subcutaneous, and intraperitoneal), transdermal, suppository, and intranasal methods. Regarding parenteral administration, it is preferably used in the form of a sterile water solution, which may comprise other substances, such as salts or glucose sufficient to make the solution isotonic to blood. Preparation of an appropriate parenteral composition under sterile conditions may be accomplished with standard pharmacological techniques well known to persons skilled in the art, and no extra creative labor is required.

In some embodiments, the pharmaceutical preparation is a composition e.g. in a form selected from the group consisting of tablets, capsules, pills, powders, granules, solutions, suspensions and emulsions, preferably for oral administration.

In some embodiments, the composition is administered in the form of gel, spray, emulsion, pastilles, dispersible tablets, tablets, enteral coated, capsules, soft capsules, granules, suspensions, microspheres, oral implants, intramuscular injection, intravenous injection, implantable injections, modified release and other pharmaceutically acceptable forms.

In some embodiments, it is preferably to provide a first analgesic agent in an extended release portion and a second analgesic agent in an immediate release portion, which can provide both a fast onset of analgesic effect and an extended duration of analgesic effect.

The present invention also provides a method for treating pain in a subject in need comprising administering to the subject an analgesic pharmaceutical preparation as described herein. In particular, the method of the invention provides synergic effects of pain relief, improved oral bioavailabilitv and less side effects.

Specifically, the method of the present invention is applicable in treating moderate to severe/deep pains, for example, associated with cancer, renal or biliary colic, migraine or vascular headaches, surgical pain and burn injury.

In some embodiments, the first analgesic agent and the second analgesic agent can be administered simultaneously or subsequently.

The present invention is further illustrated by the following examples, which are provided for the purpose of demonstration rather than limitation.

### Examples

1. Materials and Methods

1.1 Animals

Male Sprague-Dawley rats weights between 260 and 330 g were purchased from BioLASCO (Taipei, Taiwan). The rats were housed in a controlled condition (free access to food and water); 12-h light-dark cycle, temperature 22° C. and humidity 60%. All experiments were conducted in accordance with the IACUC's Protocol for the Care and Use of Animals and to treat the animals in an ethical and humane manner consistent with the law.

1.2 Drugs and Reagents

Nalbuphine was supplied by Yung-Shin Pharmaceutical Ind. Co., Ltd. (Taichung Hsien, Taiwan). SDE was supplied by Yung-Shin Pharmaceutical Ind. Co., Ltd. (Taichung Hsien, Taiwan). AAP product (a drug formulation) was supplied by China Chemical & Pharmaceutical Co., Ltd (Hsinchu Hsien, Taiwan). Acetaminophen powder were obtained from the Sigma-alorich (St. Louis, Mo., USA). Isopentyl alcohol was obtained from the Mallinckrodt baker. Inc. (Phillipsburg, N.J., USA). Hexanes was obtained from the Avantor performance materials, Inc. (Center Valley, Pa., USA). Acetonitrile and methanol were obtained from Merck (Darmstadt, Germany). Analysis was used to liquid chromatography-mass spectrometry (LC-MS) grade.

1.3 Pharmacodynamic Studies

Analgesic effects studies were used to the paw pressure test by Randall and Selitto. Nociceptive threshold is expressed in grams and measured with an analgesimeter (IITC Inc. Life Science, CA), were applied to the left hind paw of rats. All animals were tested at 15, 30, and 45 min prior to medication, to obtain an average baseline. Results were expressed as a percentage of the maximum possible effect (% MPE), according to the formula % MPA=(Test-Baseline)/(Cutoff-Baseline)×100, cut-off value: 750 g. AUC (area under curve) is independent of each individual, at each time point detection value minus own baseline and integral, then calculated the average. Negative values (% MPA and AUC) are considered as zero. The maximum peak of % MPA and Tmax are independent of each individual, then calculated the average. With 20% MPA as a baseline of valid analgesic effect, analysis of onset time of action up to 20% MPA and duration time of action over 20% MPA. Pharmacodynamic studies were conducted to compare in rats the analgesic effects of oral administration of SDE 75 mg/kg alone. AAP product (100 mg/kg) alone and combinations of SDE 75 mg/kg plus AAP product (100 mg/kg) or NAL 60 mg/kg alone. AAP product (100 mg/kg) alone and combinations of NAL 60 mg/kg plus AAP product (100 mg/kg). The anti-nociceptive thresholds were measured at 30, 60, 90, 120, 150, 180, 210, 240, 270, and 300 min after drug administration.

1.4 Pharmacokinetics Studies

The rats received orally SDE 75 mg/kg alone, AAP product (100 mg/kg) alone and combinations of SDE 75 mg/kg plus AAP product (100 mg/kg), and then blood samples were collected in different time point. The samples put into microcentrifuge tubes contain 20 μL of 20 IU heparin and isolated plasma by centrifuged in 4° C., 13300 rpm for 10 min, and stored at −80° C. until assay.

The plasma samples were extracted NAL concentrations by liquid-liquid extraction. Taking 0.1 mL aliquot of rat plasma sample added 50 μL of IS (naloxone: 2 μg/mL) before adding 50 μL of 1 N Na₂CO₃. Extraction solvent (2 mL n-hexane:isoamyl alcohol=9:1) was added and the sample was vortexed for 5 min and put in −80° C., 30 min. The upper organic phase was poured into anew glass tube and solvent was evaporated to dryness at 40° C. under a gentle stream of nitrogen (Zymark® MA, USA). The residue was reconstituted in 100 μL of the mobile phase and vortexed 30 s. Then samples was transferred to autosampler vials and analyzed by the ultra-high-performance liquid chromatography-tandem mass spectrometry (UPLC-MS/MS).

NAL were analyzed using UPLC (Waters Acquity™ Milford, Mass., USA) coupled to a Biosystems-Sciex API 3000 series triple-quadrupole mass spectrometer (Foster City, Calif., USA) with an electrospray ionization (ESI) interface. Chromatographic separation was using Waters Acquity UPLC BEH HILIC, 2.1×100 mm, 1.7 μm column. The mobile phase solvent A contain 2 mM ammonium formate and 0.1% formic acid in water and solvent B contain 2 mM ammonium formate and 0.1% formic acid in acetonitrile. The total run time was 5 min and the column temperature was maintained at 35° C. The mobile phase composition was as follows: 13% A and 87% B. The retention times of NAL were 2.89 and 2.65 min, respectively. The Q1 and Q3 of NAL were 358.1→340.1 and 328.3→310.3. Analyst 1.4.2 software (Applied Biosystems-Sciex; Foster City, Calif.) was used to collect and process the MS/MS data. NAL plasma concentration were analyzed by WinNonlin 5.3 software (Pharsight. Mountain View, Calif.). The statistical significance of data obtained from the pharmacokinetic and pharmacodynamic studies was determined using One-way ANOVA using the PRISM software.

2. Results

2.1 Analgesic Effects of Drug Formulations (Test 1)

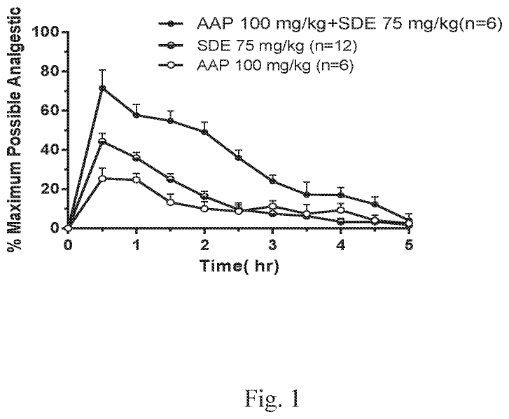

FIG. 1 shows the quantification of the anti-nociceptive effects of oral administration of AAP alone, SDE alone and combination of AAP and SDE (with % maximum possible analgesic (MPA) effect threshold value of 100%). The anti-nociceptive response was evaluated by calculating the AUC (% MPA versus time) and the duration for which the MPA was greater than 20% for each group (Table 1). Statistical analysis of the data shows that a combination of AAP and SDE exhibits synergistic analgesic effects as compared with AAP or SDE alone, and a combination of AAP and SDE exhibits synergistic analgesic effects as compared with AAP or SDE alone (p<0.005), wherein a combination of AAP and SDE exhibits relatively longest duration of action and highest AUC value, indicate superior bioavailability (p<0.005). Importantly, combination ofAAP and SDE had significantly synergic effects better than SDE and AAP alone, respectively (p<0.005). Combination of AAP and SDE did have the synergic effects of pain relief without pharmaceutic formulation adjustment.

TABLE 1
Parameters of analgesic effect of AAP alone, SDE
alone and combination of AAP and SDE in SD-rats.

| | AAP | SDE | SDE + |
|---|---|---|---|
| | alone (n = 6) | alone(n = 12) | AAP (n = 6) |
| Onset of action (min) | 46.7 ± 17.5 | 14.7 ± 1.3 | 9.1 ± 1.2^{a} |
| (20% of MPA) | | | |
| Duration of action | 77.5 ± 13.5 | 99.8 ± 10.0 | 203.5 ± 14.7^{a,b} |
| (min) (20% of MPA) | | | |
| AUC (g* hr) | 309.3 ± 68.5 | 359.4 ± 34.8 | 822.1 ± 40.8^{a,b} |
| Maximum Peak | 32.4 ± 2.7 | 46.6 ± 3.8 | 79.9 ± 5.9^{a,b} |
| (% of MPA) | | | |
| Tₘₐₓ (min) | 50.0 ± 10.0 | 37.5 ± 3.9 | 55.0 ± 12.0 |

AUC: area under curve is each time point detection value baseline and integral.
% MPA: % Maximum Possible Analgestic
Data was shown as mean ± SE.
Statistics: One-way ANOVA
^{a}p < 0.005 relative to AAP alone,
^{b}p < 0.005 relative to SDE alone;
^{c}p < 0.01 relative to AAP alone,
^{d}p < 0.01 relative to SDE alone;
^{e}p < 0.05 relative to AAP alone,
^{f}p < 0.05 relative to SDE alone,

2.2 Analgesic Effects of Various Drug Formulations (Test 2)

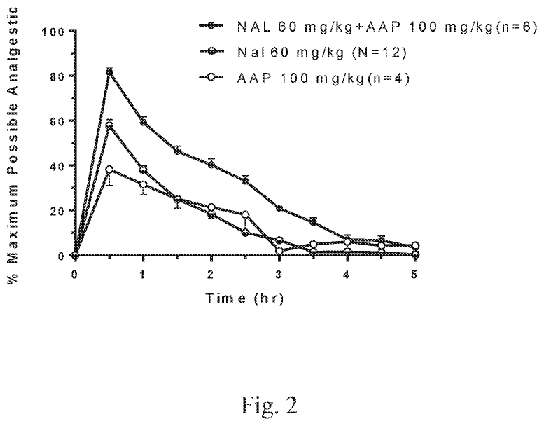

FIG. 2 shows quantification of the anti-nociceptive effects of oral administration of NAL, AAP and NAL+AAP (with % maximum possible analgesic effect (MPA) threshold value of 100%). The anti-nociceptive response was evaluated by calculating the AUC (% MPA versus time) and the duration for which the MPA was greater than 50% for each group (Table 2). Statistical analysis of the data indicated that a combination of NAL and AAP exhibits synergistic analgesic effects as compared with NAL or AAP alone (p<0.0001).

TABLE 2

| | NAL | AAP | NAL + AAP |
|---|---|---|---|
| | (n = 12) | (n = 4) | (n = 6) |
| Onset of | 24.36 ± 0.86 | — | 18.42 ± 0.38 *** |
| action (min) | | | |
| Duration of | 16.61 ± 3.92 | — | 67.43 ± 5.31 **** |
| action (min) | | | |
| AUG (g* hr) | 298.9 ± 13.98 | 274.83 ± 51.94 | 604.45 ± 23.93 **** |
| % MPA (Maxi- | 57.87 ± 2.77 | 38.26 ± 7.2 | 81.62 ± 1.72 **** |
| mum Peak) | | | |
| Tₘₐₓ (min) | 30 ± 0 | 45 ± 8.66 | 30 ± 0 ** |

Data was shown as mean ± SE.
AUC: area under curve is each time point detection value baseline and integral.
% MPA: % Maximum Possible Analgestic
Statistics: One-way ANOVA,
** p < 0.01,
*** p < 0.005,
**** p < 0.0001, compared to AAP and NAL, respectively.

1. An analgesic pharmaceutical preparation, comprising a therapeutically effective amount of a first analgesic agent which is sebacoyl dinalbuphine or its metabolites or derivatives and a therapeutically effective amount of a second analgesic agent which is acetaminophen (AAP) or its derivatives, together with one or more pharmaceutically acceptable excipients.

2. The pharmaceutical preparation of claim 1, wherein the one or more pharmaceutically acceptable excipients are selected from the group consisting of Eudragit S100, dicalcium phosphate dehydrate, Pluronic F68, hexitol, Crospovidone, Sodium starch glycolate, Aerosil 200, trichlorosucrose, menthol, Saccharin, Sodium benzoate, Glyceryl behenate, Sodium lauryl sulfate, Providone K30 and any combination thereof.

3. The pharmaceutical preparation of claim 1, which comprises a combination of the first analgesic agent and the second analgesic agent.

4. The pharmaceutical preparation of claim 1, wherein the first analgesic agent is sebacoyl dinalbuphine in the free base form or a pharmaceutically acceptable salt.

5. An analgesic pharmaceutical preparation, comprising
(i) a first analgesic composition comprising a therapeutically effective amount of a first analgesic agent which is sebacoyl dinalbuphine or its metabolites or derivatives; and
(ii) a second analgesic composition comprising a therapeutically effective amount of a second analgesic agent which is acetaminophen or its derivatives.

6. The pharmaceutical preparation of claim 5, which comprises one or more pharmaceutically acceptable excipients selected from the group consisting of Eudragit S100, dicalcium phosphate dehydrate, Pluronic F68, hexitol, Crospovidone, Sodium starch glycolate, Aerosil 200, trichlorosucrose, menthol, Saccharin, Sodium benzoate, Glyceryl behenate, Sodium lauryl sulfate, Providone K30 and any combination thereof.

7. The pharmaceutical preparation of claim 6, further comprising one or more additional excipients as a carrier as the balance.

8. The pharmaceutical preparation of claim 5, wherein the amount of first and second analgesic is 1-1,000 mg.

9. The pharmaceutical preparation of claim 5, which provides a summation/synergistic analgesic effect.

10. The pharmaceutical preparation of claim 9, wherein the summation/synergistic analgesic effect includes a higher potency, faster onset of analgesic effect and/or a longer duration of analgesic effect, when compared to the first analgesic composition or the second analgesic composition alone.

11. The pharmaceutical preparation of claim 9, wherein the summation/synergistic analgesic effect includes an increased level of one or more pharmacodynamic parameters of the first analgesic agent and the second analgesic agent in said pharmaceutical combination, when compared to a respective value of the first analgesic agent in the first analgesic composition or the second analgesic agent in the second analgesic composition.

12. The pharmaceutical preparation of claim 5, wherein the first analgesic agent is sebacoyl dinalbuphine in the free base form or a pharmaceutically acceptable salt.

13. The pharmaceutical preparation as recited in claim 1, wherein the analgesic composition is administered in the form of gel, spray, emulsion, pastilles, dispersible tablets, tablets, enteral coated, capsules, soft capsules, granules, suspensions, microspheres, oral implants, intramuscular injection, intravenous injection, implantable injections, modified release and other pharmaceutically acceptable forms.

14. A method for treating pain in a subject in need comprising administering to the subject an analgesic pharmaceutical preparation of claim 1.
